(19) United States
(12) Patent Application Publication
(10) Pub. No.: US 2011/0118458 A1
(43) Pub. Date: May 19, 2011

(54) USE OF MEDIATORS IN THE PRODUCTION OF FIBERBOARDS

(76) Inventors: Alireza Kharazipour, Goettingen (DE); Markus Christian Euring, Goettingen (DE)

(21) Appl. No.: 13/002,539

(22) PCT Filed: Aug. 19, 2009
(86) PCT No.: PCT/EP09/06019

§ 371 Date: Jan. 4, 2011

(30) Foreign Application Priority Data
Aug. 19, 2008 (DE) 10 2008 038 398.8

(51) Int. Cl. C08B 1/00; C07C 65/05; C07C 49/35; C07C 47/575; C07C 69/76; C07C 47/58; C12P 19/04

(52) U.S. Cl. 536/56; 562/475; 568/337; 568/442; 560/67; 435/101

(57) ABSTRACT

The invention relates to a novel mediator used in the production of wood composite materials that are devoid of binding agents.

The present invention pertains to the field of wood and/or composites, especially fiberboards.

Wood fiberboards are valuable materials that can be produced from a renewable raw material, namely, lignocellulose-containing substances, like wood. These wood materials are used in many processing branches as material. The furniture industry, automotive industry, packaging industry, construction industry and the like are mostly represented here. Generally, wood fiberboards are mixed with binders, shaped and then compressed under heat and pressure. So-called medium density fiberboards (MDF), high density fiberboards (HDF) and low density fiberboards (LDF) and wood fiber insulation materials are ordinarily produced from wood chips from softwood or hardwood in a defibering machine, for example, with so-called refiners (for example, according to the TMP method), and brought to the desired fiber size and fiber fineness. The wood fibers are ordinarily glued with synthetic resins in the drying method (so-called “blow line” or “blender method”) and dried to the desired wood fiber moisture content. The wood fibers are then spread mechanically in a shaping station on a conveyor belt in the form of a mat and then compressed while hot.

Another possibility is production of wood fiberboards according to the so-called wet method. In this method, the fibers are suspended with binders. The fibers have high moisture content from this method of up to 100%. For further processing, this means that the fibers must be drained and then compressed in the hot press after mat formation and pre-compaction. The wet method is used, among others, in the production of HDF boards and insulation boards.

Especially to avoid the use of formaldehyde-containing binders, in the past methods were described, for example, in DE 4305411, based on the fact that the lignin present in the wood fibers is polymerized enzymatically and therefore used as binder.

However, these methods are often technically demanding, especially time-intensive, and thus far could not be successfully used on an industrial scale.

The task was therefore to further improve the present state of known methods and at least partially overcome their drawbacks.

This task is solved by an application according to Claim 1. According to it, the use of the material is proposed, chosen from the group containing

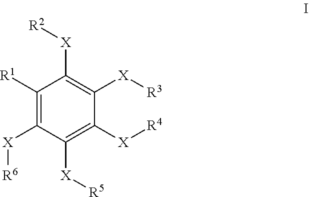

in which
R¹ is chosen from hydroxyl (—OH) and thiol (—SH);
each X is chosen independently of each other from the group containing a single bond, —CR′R″—, —CR′═CR″—, in which R′ and R″, independently of each other, are chosen from the group containing hydrogen, alkyl, aryl, cycloalkyl,
as well as
R² to R⁶, independently of each other, are chosen from the group containing hydrogen, hydroxyl, thiol, halogen, pseudohalogen, formyl, carboxy- and/or carbonyl derivatives, alkyl, long-chain alkyl, alkoxy, long-chain alkoxy, cycloalkyl, haloalkyl, aryl, arylene, haloaryl, heteroaryl, heteroarylene, heterocycloalkylene, heterocycloalkyl, haloheteroaryl, alkenyl, haloalkenyl, alkynyl, haloalkynyl, keto, ketoaryl, haloketoaryl, hetoheteroaryl, hetoalkyl, haloketoalkyl, ketoalkenyl, haloketoalkenyl, phosphoalkyl, phosphonates, phosphates, phosphine, phosphine oxide, phosphoryl, phosphoaryl, sulfonyl, sulfoalkyl, sulfoarenyl, sulfonates, sulfates, sulfones, polyethers, silylakyl, silylalkyloxy, in which, in appropriate groups, one or more non-adjacent CH₂ groups, independently of each other, can be replaced by —O—, —S—, —NH—, —NR^{∘}—, —SiR^{∘}R^{∘∘}—, —CO—, —COO—, —COO—, —COO—O—, —SO₂—, —S—CO—, —CO—S—, —CY¹═CY² or —C≡C—, and so that O and/or S atoms are not directly bonded to each other (terminal CH₃ groups, like CH₂ groups, are understood in the sense of CH₂—H),
and in which at least one group Rest R² to R⁶ is chosen from the group alkoxy, formyl, carboxy- and/or carbonyl derivatives, keto, ketoaryl, haloketoaryl, ketoheteroaryl, ketoalkyl, haloketoalkyl, ketoalkenyl, haloketoalkenyl, sulfonyl, sulfoalkyl, sulfoarenyl, sulfonates, sulfates, sulfone;

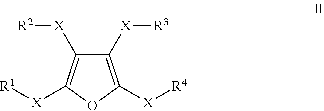

in which R¹ to R⁴, independently of each other, are chosen from the group containing hydrogen, hydroxyl, thiol, halogen, pseudohalogen, formyl, carboxy- and/or carbonyl derivatives, alkyl, long-chain alkyl, alkoxy, long-chain alkoxy, cycloalkyl, haloalkyl, aryl, arylene, haloaryl, heteroaryl, heteroarylene, heterocycloalkylene, heterocycloalkyl, haloheteroaryl, alkenyl, haloalkenyl, alkynyl, haloalkynyl, keto, ketoaryl, haloketoaryl, ketoheteroaryl, ketoalkyl, haloketoalkyl, ketoalkenyl, haloketoalkenyl, phosphoalkyl, phosphonates, phosphates, phosphine, phosphine oxide, phosphoryl, phosphoaryl, sulfonyl, sulfoalkyl, sulfoarenyl, sulfonates, sulfates, sulfones, polyether, silylalkyl, silylalkyloxy, in which, with appropriate groups, one or more non-adjacent CH₂ groups, independently of each other, could be replaced by —O—, —S—, —NH—, —NR^{∘}—, —SiR^{∘}R^{∘∘}—, —CO—, —COO—, —COO—, —COO—O—, —SO₂—, —S—CO—, —CO—S—, —CY¹═CY² or —C≡C—, and so that O and/or S atoms are not directly bonded to each other (terminal CH₃ groups, like CH₂ groups, are understood in the sense of CH₂—H),
each X, independently of each other, is chosen from the group containing a single bond, —CR′R″—, —CR′═CR″—, in which R′ and R″, independently of each other, are chosen from the group containing hydrogen, alkyl, aryl, cycloalkyl,
in which at least one group R¹ to R⁴ is chosen from the group hydroxyl and thiol, and each corresponding X represents a single bond;
and in which at least one other group R¹ to R⁴ is chosen from the group alkoxy, formyl, carboxy- and/or carbonyl derivatives, keto, ketoaryl, haloketoaryl, ketoheteroaryl, ketoalkyl, haloketoalkyl, ketoalkenyl, haloketoalkenyl, sulfonyl, sulfoalkyl, sulfoarenyl, sulfonates, sulfates, sulfone;
or mixtures of them as a mediator in the production of lignocellulose-containing molded articles, especially wood fiber and/or composite materials.

The term “mediator” is understood to mean especially low-molecular substances that act as catalyst. As an alternative or in additional, the term “mediator” is understood especially to mean low-molecular substances that are capable of cooperating with phenol-oxidizing enzymes in the desired synergistic manner.

The term “lignocellulose-containing molded articles” especially summarizes all mat-like and non-mat-like materials that contain as main ingredient ground lignocellulose-containing materials, like wood, cereal straw, hemp or flax, which are pressed under temperature and/or pressure after shaping.

The term “wood and/or composite material” is understood especially to mean materials that consist mostly of mechanically or thermomechanically ground lignocellulose-containing material, which is shaped, after sizing and/or pressed under temperature and pressure, to wood and/or composite materials.

General group definition: Within the description and claims, general groups, like alkyl, alkoxy, aryl, etc., are claimed and described. Unless otherwise described, the following groups are preferably used within the generally described groups in the context of the present invention:

alkyl: linear and branched C1-C8-alkyls,
long-chain alkyls: linear and branched C5-C20 alkyls
alkenyl: C2-C8-alkenyl,
cycloalkyl: C3-C8-cycloalkyl,
alkoxy: C1-C6-alkoxy,
long-chain alkoxy: linear and branched C5-C20 alkoxy
alkylene: chosen from the group containing:
methylene; 1,1-ethylene; 1,2-ethylene; 1,1-propylidene; 1,2-propylene; 1,3-propylene; 2,2-propylidene; butan-2-ol-1,4-diyl; propan-2-ol-1,3-diyl; 1,4-butylene; cyclohexane-1,1-diyl; cyclohexane-1,2-diyl; cyclohexane-1,3-diyl; cyclohexane-1,4-diyl; cyclopentane-1,1-diyl; cyclopentane-1,2-diyl; and cyclopentane-1,3-diyl,
aryl: chosen from aromatics with molecular weight under 300 Da.
arylene: chosen from the group containing: 1,2-phenylene; 1,3-phenylene; 1,4-phenylene; 1,2-naphthalenylene; 1,3-naphthalenylene; 1,4-naphtalenylene; 2,3-naphthalenylene; 1-hydroxy-2,3-phenylene; 1-hydroxy-2,4-phenylene; 1-hydroxy-2,5-phenylene; and 1-hydroxy-2,6-phenylene,
carboxy derivatives: the groups —COXR₁, in which X represents NH or O and R₁ is chosen from the group containing alkyl, aryl, cycloalkyl, heteroaryl, heterocycloalkyl.
heteroaryl: chosen from the group containing: pyridinyl; pyrimidinyl; pyrazinyl; triazolyl; pyridazinyl; 1,3,5-triazinyl; quinolinyl; isoquinolinyl; quinoxalinyl; imidazolyl; pyrazolyl; benzimidazolyl; thiazolyl; oxazolidinyl; pyrrolyl; thiophenyl; carbazolyl; indolyl; and isoindolyl, in which heteroaryl can be bonded to the compound by each atom in the ring of the selected heteroaryl.
heteroarylene: chosen from the group containing: pyridindiyl; quinolindiyl; pyrazodiyl; pyrazoldiyl; triazolediyl; pyrazindiyl, thiophendiyl; and imidazolediyl, in which the heteroarylene functions as a bridge in the compound via any atom in the ring of the selected heteroaryl, with particular preference: pyridine-2,3-diyl; pyridine-2,4-diyl; pyridine-2,5-diyl; pyridine-2,6-diyl; pyridine-3,4-diyl; pyridine-3,5-diyl; quinoline-2,3-diyl; quinoline-2,4-diyl; quinoline-2,8-diyl; isoquinoline-1,3-diyl; isoquinoline-1,4-diyl; pyrazole-1,3-diyl; pyrazole-3,5-diyl; triazole-3,5-diyl; triazole-1,3-diyl; pyrazine-2,5-diyl; and imidazole-2,4-diyl, thiophene-2,5-diyl, thiophene-3,5-diyl; a C1-C6-heterocycloalkyl, chosen from the group containing: piperidinyl; piperidine; 1,4-piperazine, tetrahydrothiophene; tetrahydrofuran; 1,4,7-triazacyclononane; 1,4,8,11-tetraazacyclotetradecane; 1,4,7,10,13-pentaazacyclopentadecane; 1,4-diaza-7-thia-cyclononane; 1,4-diaza-7-oxa-cyclononane; 1,4,7,10-tetraazacyclododecane; 1,4-dioxane; 1,4,7-trithia-cyclononane; pyrrolidine; and tetrahydropyran, in which the heteroaryl can be bonded to C1-C6-alkyl via any atom in the ring of the selected heteroaryl.
heterocycloalkylene: chosen from the group containing: piperidin-1,2-ylene; piperidin-2,6-ylene; piperidin-4,4-ylidene; 1,4-piperazin-1,4-ylene; 1,4-piperazin-2,3-ylene; 1,4-piperazin-2,5-ylene; 1,4-piperazin-2,6-ylene; 1,4-piperazin-1,2-ylene; 1,4-piperazin-1,3-ylene; 1,4-piperazin-1,4-ylene; tetrahydrothiophen-2,5-ylene; tetrahydrothiophen-3,4-ylene; tetrahydrothiophen-2,3-ylene; tetrahydrofuran-2,5-ylene; tetrahydrofuran-3,4-ylene; tetrahydrofuran-2,3-ylene; pyrrolidin-2,5-ylene; pyrrolidin-3,4-ylene; pyrrolidin-2,3-ylene; pyrrolidin-1,2-ylene; pyrrolidin-1,3-ylene; pyrrolidin-2,2-ylidene; 1,4,7-triazacyclonon-1,4-ylene; 1,4,7-triazacyclonon-2,3-ylene; 1,4,7-triazacyclonon-2,9-ylene; 1,4,7-triazacyclonon-3,8-ylene; 1,4,7-triazacyclonon-2,2-ylidene; 1,4,8,11-tetraazacyclotetradec-1,4-ylene; 1,4,8,11-tetraazacyclotetradec-1,8-ylene; 1,4,8,11-tetraazacyclotetradec-2,3-ylene; 1,4,8,11-tetraazacyclotetradec-2,5-ylene; 1,4,8,11-tetraazacyclotetradec-1,2-ylene; 1,4,8,11-tetraazacyclotetradec-2,2-ylidene; 1,4,7,10-tetraazacyclododec-1,4-ylene; 1,4,7,10-tetraazacyclododec-1,7-ylene; 1,4,7,10-tetraazacyclododec-1,2-ylene; 1,4,7,10-tetraazacyclododec-2,3-ylene; 1,4,7,10-tetraazacyclododec-2,2-ylidene; 1,4,7,10,13 pentaazacyclopentadec-1,4-ylene; 1,4,7,10,13-pentaazacyclopentadec-1,7-ylene; 1,4,7,10,13-pentaazacyclopentadec-2,3-ylene; 1,4,7,10,13-pentaazacyclopentadec-1,2-ylene; 1,4,7,10,13-pentaazacyclopentadec-2,2-ylidene; 1,4-diaza-7-thia-cyclonon-1,4-ylene; 1,4-diaza-7-thia-cyclonon-1,2-ylene; 1,4-diaza-7thia-cyclonon-2,3-ylene; 1,4-diaza-7-thia-cyclonon-6,8-ylene; 1,4-diaza-7-thia-cyclonon-2,2-ylidene; 1,4-diaza-7-oxacyclonon-1,4-ylene; 1,4-diaza-7-oxa-cyclonon-1,2-ylene; 1,4diaza-7-oxa-cyclonon-2,3-ylene; 1,4-diaza-7-oxa-cyclonon-6,8-ylene; 1,4-diaza-7-oxa-cyclonon-2,2-ylidene; 1,4-dioxan-2,3-ylene; 1,4-dioxan-2,6-ylene; 1,4-dioxan-2,2-ylidene; tetrahydropyran-2,3-ylene; tetrahydropyran-2,6-ylene; tetrahydropyran-2,5-ylene; tetrahydropyran-2,2-ylidene; 1,4,7-trithia-cyclonon-2,3-ylene; 1,4,7-trithia-cyclonon-2,9-ylene; and 1,4,7-trithia-cyclonon-2,2-ylidene,
heterocycloalkyl: chosen from the group containing: pyrrolinyl; pyrrolidinyl; morpholinyl; piperidinyl; piperazinyl; hexamethylene imine; 1,4-piperazinyl; tetrahydrothiophenyl; tetrahydrofuranyl; 1,4,7-triazacyclononanyl; 1,4,8,11-tetraazacyclotetradecanyl; 1,4,7,10,13-pentaazacyclopentadecanyl; 1,4-diaza-7-thiacyclononanyl; 1,4-diaza-7-oxa-cyclononanyl; 1,4,7,10-tetraazacyclododecanyl; 1,4-dioxanyl; 1,4,7-trithiacyclononanyl; tetrahydropyranyl; and oxazolidinyl, in which the heterocycloalkyl can be bonded to the compound by any atom in the ring of a selected heterocycloalkyl.
halogen: chosen from the group containing: F; Cl; Br and I,
haloalkyl: chosen from the group containing mono, di, tri-, poly and perhalogenated linear and branched C1-C8-alkyl
pseudohalogen: chosen from the group containing —CN, —SCN, —OCN, N3, —CNO, —SeCN

Unless otherwise mentioned, the following groups are more preferred groups within the general group definition:

alkyl: linear and branched C1-C6-alkyl, more preferably methyl and ethyl
long-chain alkyl: linear and branched C5-C10 alkyl, preferably C6-C8 alkyl
alkenyl: C3-C6-alkenyl,
cycloalkyl: C6-C8-cycloalkyl,
alkoxy: C1-C4-alkoxy, more preferably methoxy and ethoxy
long-chain alkoxy: linear and branched C5-C10 alkoxy, preferably linear C6-C8 alkoxy
alkylene: chosen from the group containing: methylene; 1,2-ethylene; 1,3-propylene; butan-2-ol-1,4-diyl; 1,4-butylene; cyclohexane-1,1-diyl; cyclohexane-1,2-diyl; cyclohexane-1,4-diyl; cyclopentane-1,1-diyl; and cyclopentane-1,2-diyl, aryl: chosen from the group containing: phenyl; biphenyl; naphthalenyl; anthracenyl; and phenanthrenyl,
arylene: chosen from the group containing: 1,2-phenylene; 1,3-phenylene; 1,4-phenylene; 1,2-naphthalenylene; 1,4-naphthalenylene; 2,3-naphthalenylene and 1-hydroxy-2,6-phenylene,
heteroarylene: thiophene, pyrrole, pyridine, pyridazine, pyrimidine, indole, thienothiophene
halogen: chosen from the group containing: Br and Cl, especially Br

It surprisingly turned out that the use of such a material according to the invention in the production of lignocellulose-containing molded articles is advantageous. Use of a material according to the present invention is particularly worthwhile in most applications with one or more of the following advantages:
Production of lignocellulose-containing molded articles is possible more quickly and simply
Formaldehyde-containing binders can largely be eliminated, sometimes even entirely avoided.
During production, further processing and subsequent use, no harmful emissions are produced.
The material according to the invention forms no toxic degradation product, so that use is safe
Products so produced can be recycled without problem.

According to a preferred variant, the material according to the invention contains at least one material with the following structure

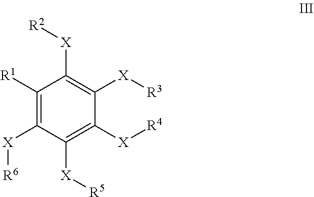

in which
R¹ is chosen from hydroxyl (—OH) and thiol (—SH);
R⁴ is chosen from the group containing alkoxy, formyl, carboxy- and/or carbonyl derivatives, keto, ketoaryl, haloketoaryl, ketoheteroaryl, ketoalkyl, haloketoalkyl, ketoalkenyl, haloketoalkenyl, sulfonyl, sulfoalkyl, sulfoarenyl, sulfonates, sulfates, sulfone;
each X, independently of each other, is chosen from the group containing a single bond, —CR′R″—, —CR′═CR″—, in which R′ and R″, independently of each other, are chosen from the group containing hydrogen, alkyl, aryl, cycloalkyl,
as well as
R², R³, R⁵ and R⁶, independently of each other, are chosen from the group containing hydrogen, hydroxyl, thiol, halogen, pseudohalogen, formyl, carboxy- and/or carbonyl derivatives, alkyl, long-chain alkyl, alkoxy, long-chain alkoxy, cycloalkyl, haloalkyl, aryl, arylene, haloaryl, heteroaryl, heteroarylene, heterocycloalkylene, heterocycloalkyl, haloheteroaryl, alkenyl, haloalkenyl, alkynyl, haloalkynyl, keto, ketoaryl, haloketoaryl, ketoheteroaryl, ketoalkyl, haloketoalkyl, ketoalkenyl, haloketoalkenyl, phosphoalkyl, phosphonates, phosphates, phosphine, phosphine oxide, phosphoryl, phosphoaryl, sulfonyl, sulfoalkyl, sulfoarenyl, sulfonates, sulfates, sulfones, polyether, silylalkyl, silylalkyloxy, in which, with appropriate groups, one or more non-adjacent CH₂ groups, independently of each other, can be replaced by —O—, —S—, —NH—, —NR^{∘}—, —SiR^{∘}R^{∘∘}—, —CO—, —COO—, —COO—, —COO—O—, —SO₂—, —S—CO—, —CO—S—, —CY¹═CY² or —C≡C—, and so that O and/or S atoms are not directly bonded to each other (terminal CH₃ groups, like CH₂ groups, are understood in the sense of CH₂—H).

These materials have proven particularly valuable in practice. Without being restricted to this idea, it is attributed to the fact that the hydroxy/thiol function in position 4 is an activating group.

According to a preferred variant of the invention, the material according to the invention contains no nitrogen. This has proven advantageous, since it can be ensured in simple fashion that no undesired degradation products (like nitrous gases, etc.) form in the production of lignocellulose-containing molded articles.

According to a preferred variant, the at least one material is chosen from the group containing
Hydroxybenzoic acid, preferably 4-hydroxybenzoic acid, as well as its esters, preferably alkyl- and aryl esters
Hydroxyfuranoic acid, preferably 2-hydroxyfuran-5-carboxylic acid and/or 3-hydroxyfuran-5-carboxylic acid, as well as its esters, preferably alkyl- and aryl esters
Hydroxycinnamic acid, preferably 4-hydroxycinnamic acid, as well as its esters, preferably alkyl- and aryl esters
Compounds of general structure IV

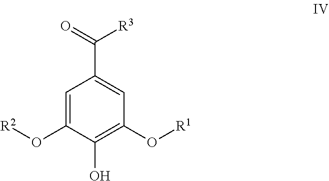

in which R¹, R² and R³, independently of each other, are chosen from hydrogen, alkyl (preferably methyl and/or ethyl), cycloalkyl and aryl. Acetosyringone (R¹, R², R³=methyl), syringaldehyde (R¹, R²=methyl, R³═H) are especially preferred.
Compounds of general structure V

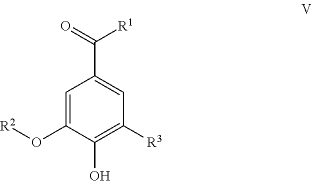

in which R¹, R² and R³, independently of each other, are chosen from hydrogen, alkyl (preferably methyl and/or ethyl), cycloalkyl and aryl, acetovanillone (R¹, R²=methyl, R³═H), vanillin (R¹=methyl, R², R³═H), ethylvanillin (R¹=ethyl, R², R³═H) are especially preferred.
Compounds of general structure VI

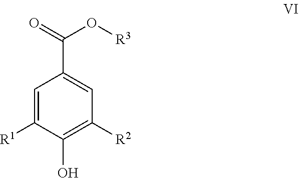

in which R¹, R² and R³, independently of each other, are chosen from hydrogen, alkoxy (preferably methoxy and/or ethoxy), alkyl (preferably methyl and/or ethyl), cycloalkyl and aryl. Methyl syringate (R¹, R²=methoxy, R³=methyl), vanillic acid (R¹, R³═H, R²=methoxy) are particularly preferred.
Compounds of general structure VII

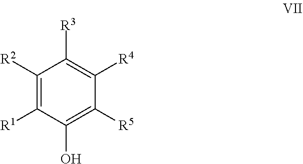

in which R¹, R², R³, R⁴ and R⁵, independently of each other, are chosen from hydrogen, hydroxy, alkyl (preferably methyl and/or ethyl), alkoxy (preferably methoxy and/or ethoxy), cycloalkyl and aryl. 2-6-dimethylphenol (R¹, R⁵=methyl, R², R³, R⁴═H), 2,6-dimethoxyphenol (R¹, R⁵=methoxy, R², R³, R⁴═H), 3-methoxyphenol (R²=methoxy, R¹, R³, R⁴, R⁵═H), 2-hydroxybiphenyl (R¹=phenyl, R², R³, R⁴, R⁵═H), 3-hydroxybiphenyl (R²=phenyl, R¹, R³, R⁴, R⁵═H), 4-hydroxybiphenyl (R³=phenyl, R¹, R², R⁴, R⁵═H), catechol (R¹=hydroxy, R², R³, R⁴, R⁵═H), guaiacol (R¹=methoxy, R², R³, R⁴, R⁵═H), 2,4,6-trimethoxyphenol (R¹, R³, R⁵=methoxy, R², R⁴═H) are particularly preferred.
Compounds of general structure VIII

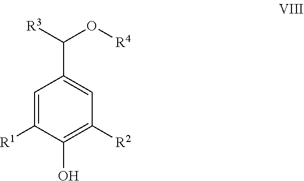

in which R¹, R², R³ and R⁴, independently of each other, are chosen from hydrogen, alkoxy (preferably methoxy and/or ethoxy), alkyl (preferably methyl and/or ethyl), cycloalkyl and aryl. Vanillyl alcohol (R¹, R³, R⁴═H, R²=methoxy) is particularly preferred.
Compounds of general structure IX

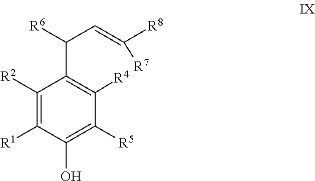

in which R¹, R², R³, R⁴, R⁵, R⁶, R⁷ and R⁸, independently of each other, are chosen from hydrogen, alkyl (preferably methyl and/or ethyl), alkoxy (preferably methoxy and/or ethoxy), cycloalkyl and aryl. 2,6-dimethoxy-4-allylphenol (R¹, R⁵=methoxy, R², R³, R⁴, R⁶, R⁷, R⁸═H), 3-methoxy-4-allylphenol (R²=methoxy, R¹, R³, R⁴, R⁵, R⁶, R⁷, R⁸═H) are particularly preferred.
Phenolphthalein, dichloroindophenol
Hydroxyanthranilic acid, preferably 3-hydroxyanthranilic acid and its esters, preferably alkyl- and/or aryl esters
Hydroxybenzyl alcohol, preferably 2- and/or 4-hydroxybenzyl alcohol
   or their mixtures.

According to a preferred variant of the invention, the material is used together with at least one phenol oxidizing enzyme, preferably chosen from the group laccases, Mg-peroxidases, lignin peroxidases, ligninases, bilirubin oxidases, catechol oxidases or their mixtures.

According to a preferred variant of the invention, the ratio between the material and the enzyme is between ≧0.5 U/mL enzyme per 1 mM material (optionally the sum of materials) and ≦40 U/mL enzyme per 1 mM material.

The activity of the enzyme (in units per milliliter U/mL) is then measured in so-called “ABTS units” according to MATSUMURA, E.; YAMAMOTO, E.; NUMATA, A.; KAWANO, T.; SHIN, T.; MURAO, S. (1986): Structures of the Laccase-catalysed Oxidation Products of Hydroxybenzoic Acids in the presence of ABTS. Japan Society for Bioscience, Tiotech. and Agrochem., Agric. Biol. Chem. 50 (5), pp. 1355-1357.

This has proven itself, in particular, in practice. With particular preference, the ratio is from ≧1 U/mL enzyme per 1 mM material to ≦30 U/mL enzyme per 1 mM material, even more preferably ≧10 U/mL enzyme per 1 mM material to ≦20 U/mL enzyme per 1 mM material.

According to a preferred variant of the invention, the lignocellulose-containing molded article is binder-free.

The term “binder-free” according to the present invention then includes especially the fact that no synthetic or natural binders (for example, amino plastics, phenoplastics, isocyanates, etc., proteins, tannins, starch, etc.) are used and/or the percentage of this binder in the finished lignocellulose-containing molded article is less than 1 wt. %. It surprisingly turned out that by the use according to the invention, other binders can (essentially) be dispensed with in most applications.

According to a preferred variant of the invention, the lignocellulose-containing molded articles are produced from lignin-containing fibers, wood fibers being particularly preferred.

The term “fibers” then especially means lignin-containing fibers with a length of ≧0.5 mm to ≦10 mm and a fiber diameter from ≧0.05 mm to ≦3 mm Fibers with a length from ≧1 mm to ≦6 mm and a fiber diameter of ≧0.1 mm to ≦1 mm are particularly preferred.

The task according to the invention is also solved by methods for production of lignocellulose-containing molded articles, especially wood and/or composite materials, comprising the steps
a) Mixing of at least one precursor material with a solution containing at least one mediator and at least one phenol-oxidizing enzyme
b) Immediate mechanical and/or thermomechanical deformation.

The term “immediately” means and/or includes especially an incubation time of <30 min, preferably <20 min, even more preferably <10 min. According to a preferred variant of the invention, step b) is carried out without incubation time.

The term “incubation time” is understood to mean that after application of the enzyme media mixture, a certain time (=the incubation time) is waited, which is independent of the rest of the production process. If no incubation time is present or necessary, step b) is therefore conducted without delay according to step a), which would not be attributed to the other specific application.

It surprisingly turned out that such a method in the production of lignocellulose-containing molded articles is advantageous. The method according to the present invention offers one or more of the following advantages in most applications:
Production of the lignocellulose-containing molded article is possible more quickly and simply.
The raw materials are permanently available.
Formaldehyde-containing binders can largely be avoided, sometimes even entirely avoided.
During production, further processing and subsequent use, no harmful emissions are produced.
The material according to the invention forms no toxic degradation product, so that use is safe.
The produced products can be recycled without problem.
The use offers the wood material companies opportunities to establish environmentally accepted production methods.
The product is produced independently of rising crude oil price.
Sale of formaldehyde-free products not hazardous to health finds broad acceptance among consumers.

The term “precursor material” is understood to mean, especially according to a preferred variant of the invention, lignin-containing fibers, especially wood fibers, but also hemp fibers, flax fibers, jute fibers, fibers from cotton stems, fibers from cereal straw, fibers of certain sweet grasses, etc.

The term fiber then especially means lignin-containing fibers with a length of ≧0.5 mm to ≦10 mm and a fiber diameter from ≧0.05 mm to ≦3 mm Fibers with a length of ≧1 mm to ≦6 mm and a fiber diameter from ≧0.1 mm to ≦1 mm are particularly preferred.

According to a preferred variant of the present invention, a material as described above is used as mediator.

According to a preferred variant of the invention, the mediator in step a) is used together with at least one phenol oxidizing enzyme, chosen from the group laccases, Mg-peroxidases, lignin peroxidases, ligninases or their mixtures.

According to a preferred variant, the concentration (in U/mL) of at least one enzyme (or the combined concentration of enzymes) is ≧50 U/mL to ≦400 U/mL. This has proven itself, in particular, in practice. The concentration with particular preference lies at ≧100 U/mL to ≦300 U/mL, even more preferably ≧150 U/mL to ≦250 U/mL.

According to a preferred variant of the invention, the ratio between mediator and at least one enzyme is between ≧0.5 U/mL enzyme per 1 mM mediator (optionally the sum of materials) to ≦40 U/mL enzyme per 1 mM mediator.

This has proven itself, in particular, in practice. The ratio with particular preference is ≧1 U/mL enzyme per 1 mM mediator to ≦30 U/mL enzyme per 1 mM mediator, even more preferably ≧10 U/mL enzyme per 1 mM mediator to ≦20 U/mL enzyme per 1 mM mediator.

The present invention also pertains to a lignocellulose-containing molded article, especially a wood and/or composite material, especially a fiberboard produced using one of the materials according to the invention and/or according to the method of the invention.

The lignocellulose-containing molded article is especially a wood and/or composite material, especially a fiberboard free of binder.

The lignocellulose-containing molded article according to the invention can be used in a number of applications, especially (but not restricted to):
MDF boards for non-bearing purposes in the dry interior area (furniture and interior construction)
Veneer boards: Veneer—MDF support board—veneer
MDF under decorative paper
MDF under surface coating
HDF for laminate floors, parquet floors
Insulation boards, for example, heat- and footfall sound insulating material
LDF boards
Molded parts, for example, in the sanitary field
Moldings, for example, for automotive internal paneling

The aforementioned components to be used according to the invention, described and claimed in the practical examples, are not subject to special exceptions in size, shaping, choice of material and technical conception, so that the selection criteria known in the area of application can be used without restriction.

Additional details, features and advantages of the object of the invention are apparent from the dependent claims and the following description of corresponding examples and drawings, in which several practical examples are lignocellulose-containing molded articles according to the invention. In the drawings, which refer to the examples:

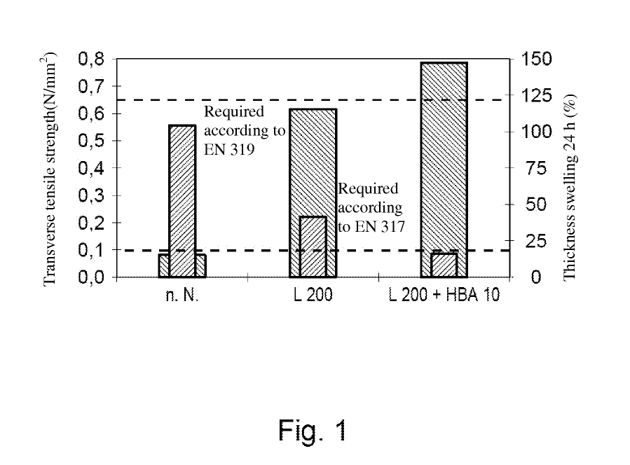

FIG. 1 shows the mechanical-processing properties (transverse tensile strength and thickness swelling) of a wood fiberboard according to the present invention and two boards according to comparative examples; as well as

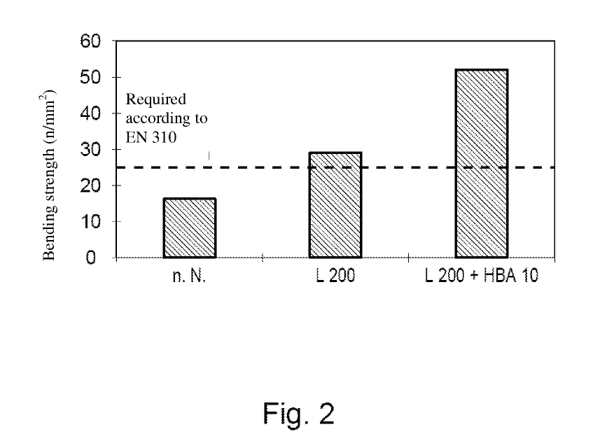

FIG. 2 shows the bending strengths of the boards according to FIG. 1.

### EXAMPLE I

The invention is shown below according to an example to be understood as purely illustrative.

For this purpose, 4-hydroxybenzoic acid was used as mediator and commercial laccase (Novozyme, Denmark) as enzyme and an MDF fiberboard, produced according to the following method from wood fibers (TMP fibers, consisting of pine, spruce or beechwood with a length of 1.23 mm to 5.12 mm and a fiber diameter from 0.11 mm to 0.87 mm):

A buffer mixture was set at 200 U/mL laccase and 10 mm 4-hydroxybenzoic acid (L 200+10 mM HBA).

The wood fibers were then sprayed with the laccase mediator solution in a uniaxial mixer (blender method). This is a spray method, ordinarily known as a drying method. 1 L solution was ordinarily used on 1 kg wood fibers.

The wood fibers were dried after spraying with laccase mediator buffer mixture directly after spraying in the mixer with the tubular drying unit to 10%-14% moisture. The dryer input temperature was then 100° C.-120° C., the dryer output temperature 40° C. After drying, spreading to a fiber mat and compression to MDF boards occurred.

A hydrophobing agent was not used in production of MDF boards.

All produced fiberboards were ground and cut to size after a cooling phase (at least 4 hours), in order to be tested according to the mechanical-processing property test according to EN Standards 310, 317 and 319. Testing of the bending and transverse tensile strengths occurred in a Zwick-Roell test machine, testing of the crude densities by means of the crude density profile measurement device DA-X from GreCon.

Wood fiberboards, in which the native wood fibers were sprayed with a buffer mixture of denatured laccase (dead-autoclaved=inactive enzyme) and without mediator, and compressed MDF boards served as reference boards. For comparative purposes, MDF boards were also produced, whose fibers had been sprayed beforehand only with laccase buffer mixture (L 200) without mediator.

FIG. 1 shows the transverse tensile strength (wide columns) and thickness swelling (thin columns) for the wood fiberboard according to the invention (L 200+HBA 10), as well as the mentioned comparative examples, i.e., a reference wood fiberboard and a wood fiberboard only with laccase (L 200).

In considering the figure, it is conspicuous that in the board produced according to the invention (L 200+10 mM HBA), an average transfer tensile strength of 0.79 N/mm² was reached. This means a distinct fulfillment of EN Standard 319, in which 0.65 N/mm² is required. The thickness swelling value, at 16.8% in the board L 200+HBA 10, could also be reduced just below the maximum swelling value of 17% required according to EN 317. In the boards L 200 (only with laccase), the transverse tensile strength at 0.62 N/mm² was too low and the swelling value at 42% too high to satisfy the standards.

No standards were satisfied in the reference wood fiberboards (with denatured=inactive lactase). The transverse tensile strength was 0.08 N/mm², the thickness swelling 105%.

In additional to determining the transverse tensile strengths and thickness swellings, the bending strengths of the aforementioned MDF boards were also tested (FIG. 2).

As s apparent in FIG. 2, the two laccase and laccase-mediator-bonded MDF boards satisfy the EN Standard 310 required for bending strength. It is apparent that the bending strength, during use of 10 mM HBA with about 52 N/mm², is significantly higher than without mediator (29 N/mm² in the sample L 200). Very low bending strengths were measured in the reference wood fiberboards.

### EXAMPLE II TO VI

Similar to example I, additional MDF fiberboards were then produced according to the present invention. The mediators II to VI mentioned in Table I were then used.

TABLE I

| Mediators | Example No. | Bending strength (N/mm²) |
|---|---|---|
| Acetosyringone | II | 25 |
| Syringaldehyde | III | 23 |
| Methyl syringate | IV | 21 |
| Vanillin | V | 17.7 |
| Vanillic acid | VI | 18 |

The bending strengths (in N/mm²) were also measured similar to above. The employed mediators also reached very good results.

It can be concluded from these results that during enzymatic activation of wood fibers with laccase and mediator according to the invention, no incubation time is necessary, in order to produce MDF boards that satisfy the standards. The laccase-mediator system therefore produces a significant chemical and physical effect on the wood fibers.

1. Use of a material, chosen from the group containing 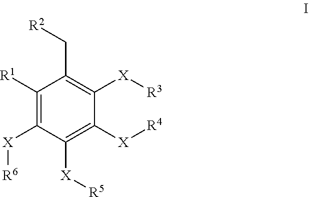
in which
R¹ is chosen from hydroxyl (—OH) and thiol (—SH);
each X, independently of each other, is chosen from the group containing a single bond, —CR′R″—, —CR′═CR″—, in which R′ and R″, independently of each other, are chosen from the group containing hydrogen, alkyl, aryl, cycloalkyl,
as well as
R² to R⁶, independently of each other, are chosen from the group containing hydrogen, hydroxyl, thiol, halogen, pseudohalogen, formyl, carboxy- and/or carbonyl derivatives, alkyl, long-chain alkyl, alkoxy, long-chain alkoxy, cycloalkyl, haloalkyl, aryl, arylene, haloaryl, heteroaryl, heteroarylene, heterocycloalkylene, heterocycloalkyl, haloheteroaryl, alkenyl, haloalkenyl, alkynyl, haloalkynyl, keto, ketoaryl, haloketoaryl, hetoheteroaryl, hetoalkyl, haloketoalkyl, ketoalkenyl, haloketoalkenyl, phosphoalkyl, phosphonates, phosphates, phosphine, phosphine oxide, phosphoryl, phosphoaryl, sulfonyl, sulfoalkyl, sulfoarenyl, sulfonates, sulfates, sulfones, polyethers, silylakyl, silylalkyloxy, in which, in appropriate groups, one or more non-adjacent CH₂ groups, independently of each other, can be replaced by —O—, —S—, —NH—, —NR^{∘}—, —SiR^{∘}R^{∘∘}—, —CO—, —COO—, —COO—, —COO—O—, —SO₂—, —S—CO—, —CO—S—, —CY¹═CY² or —C≡C—, and specifically so that O and/or S atoms are not directly bonded to each other (terminal CH₃ groups, like CH₂ groups, are understood in the sense of CH₂—H),
and in which at least one group Rest R² to R⁶ is chosen from the group alkoxy, formyl, carboxy- and/or carbonyl derivatives, keto, ketoaryl, haloketoaryl, ketoheteroaryl, ketoalkyl, haloketoalkyl, ketoalkenyl, haloketoalkenyl, sulfonyl, sulfoalkyl, sulfoarenyl, sulfonates, sulfates, sulfone; 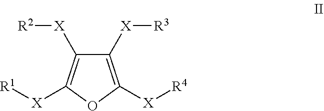
in which R¹ to R⁴, independently of each other, are chosen from the group containing hydrogen, hydroxyl, thiol, halogen, pseudohalogen, formyl, carboxy- and/or carbonyl derivatives, alkyl, long-chain alkyl, alkoxy, long-chain alkoxy, cycloalkyl, haloalkyl, aryl, arylene, haloaryl, heteroaryl, heteroarylene, heterocycloalkylene, heterocycloalkyl, haloheteroaryl, alkenyl, haloalkenyl, alkynyl, haloalkynyl, keto, ketoaryl, haloketoaryl, ketoheteroaryl, ketoalkyl, haloketoalkyl, ketoalkenyl, haloketoalkenyl, phosphoalkyl, phosphonates, phosphates, phosphine, phosphine oxide, phosphoryl, phosphoaryl, sulfonyl, sulfoalkyl, sulfoarenyl, sulfonates, sulfates, sulfones, polyether, silylalkyl, silylalkyloxy, in which, with appropriate groups, one or more non-adjacent CH₂ groups, independently of each other, could be replaced by —O—, —S—, —NH—, —NR^{∘}—, —SiR^{∘}R^{∘∘}—, —CO—, —COO—, —COO—, —COO—O—, —SO₂—, —S—CO—, —CO—S—, —CY¹═CY² or —C≡C—, and specifically so that O and/or S atoms are not directly bonded to each other (terminal CH₃ groups, like CH₂ groups, are under-stood in the sense of CH₂—H),
each X, independently of each other, is chosen from the group containing a single bond, —CR′R″—, —CR′═CR″—, in which R′ and R″, independently of each other, are chosen from the group containing hydrogen, alkyl, aryl, cycloalkyl,
in which at least one group R¹ to R⁴ is chosen from the group hydroxyl and thiol, and each corresponding X represents a single bond;
and in which at least one other group R¹ to R⁴ is chosen from the group alkoxy, formyl, carboxy- and/or carbonyl derivatives, keto, ketoaryl, haloketoaryl, ketoheteroaryl, ketoalkyl, haloketoalkyl, ketoalkenyl, haloketoalkenyl, sulfonyl, sulfoalkyl, sulfoarenyl, sulfonates, sulfates, sulfone;
or mixtures thereof as a mediator in the production of lignocellulose-containing molded articles, especially wood fiber and/or composite materials.

2. Use according to claim 1, in which the material includes a material with the following structure III: 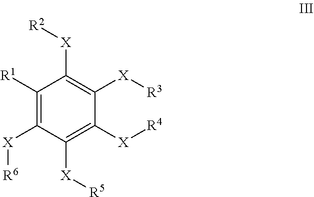
in which
R¹ is chosen from hydroxyl (—OH) and thiol (—SH);
R⁴ is chosen from the group containing alkoxy, formyl, carboxy- and/or carbonyl derivatives, keto, ketoaryl, haloketoaryl, ketoheteroaryl, ketoalkyl, haloketoalkyl, ketoalkenyl, haloketoalkenyl, sulfonyl, sulfoalkyl, sulfoarenyl, sulfonates, sulfates, sulfone;
each X, independently of each other, is chosen from the group containing a single bond, —CR′R″—, —CR′═CR″—, in which R′ and R″, independently of each other, are chosen from the group containing hydrogen, alkyl, aryl, cycloalkyl,
as well as
R², R³, R⁵ and R⁶, independently of each other, are chosen from the group containing hydrogen, hydroxyl, thiol, halogen, pseudohalogen, formyl, carboxy- and/or carbonyl derivatives, alkyl, long-chain alkyl, alkoxy, long-chain alkoxy, cycloalkyl, haloalkyl, aryl, arylene, haloaryl, heteroaryl, heteroarylene, heterocycloalkylene, heterocycloalkyl, haloheteroaryl, alkenyl, haloalkenyl, alkynyl, haloalkynyl, keto, ketoaryl, haloketoaryl, ketoheteroaryl, ketoalkyl, haloketoalkyl, ketoalkenyl, haloketoalkenyl, phosphoalkyl, phosphonates, phosphates, phosphine, phosphine oxide, phosphoryl, phosphoaryl, sulfonyl, sulfoalkyl, sulfoarenyl, sulfonates, sulfates, sulfones, polyether, silylalkyl, silylalkyloxy, in which, with appropriate groups, one or more non-adjacent CH₂ groups, independently of each other, can be replaced by —O—, —S—, —NH—, —NR^{∘}—, —SiR^{∘}R^{∘∘}—, —CO—, —COO—, —OCO—, —OCO—O—, —SO₂—, —S—CO—, —CO—S—, —CY¹═CY² or —C≡C—, and specifically so that O and/or S atoms are not directly bonded to each other (terminal CH₃ groups, like CH₂ groups, are understood in the sense of CH₂—H).

3. Use according to claim 1, in which the material contains no nitrogen.

4. Use according to claim 1, in which the material is used together with at least one phenol oxidizing enzyme, preferably chosen from the group laccases, Mg-peroxidases, lignin peroxidases, ligninases, bilirubin oxidases, catechol oxidases or their mixtures

5. Use according to claim 1, in which the ratio between material and enzyme is ≧0.5 U/mL enzyme per 1 mM material to ≦40 U/mL enzyme per 1 mM material.

6. Use according to claim 1, in which the lignocellulose-containing molded article is binder-free.

7. Method for production of lignocellulose-containing molded articles, especially wood and/or composite materials, comprising the steps:
a) Mixing of at least one precursor material with a solution containing at least one mediator and at least one phenol oxidizing enzyme
b) Immediate mechanical and/or thermomechanical deformation.

8. Method for production of lignocellulose-containing molded articles, especially wood and/or composite materials, using a material according to claim 1, comprising the steps:
a) Mixing of at least one precursor material with a solution containing at least one mediator and at least one phenol oxidizing enzyme
b) Immediate mechanical and/or thermomechanical deformation.

9. Method according to claim 7, in which the concentration in step a) (in U/mL) of the at least one enzyme is ≧50 U/mL to ≦400 U/mL.

10. Lignocellulose-containing molded articles, especially a wood and/or composite material, produced using the material according to claim 8.

11. Binder-free lignocellulose-containing molded articles according to claim 10.

12. Lignocellulose-containing molded articles according to claim 10, chosen from the group containing:
MDF boards for non-bearing purposes in the dry interior area
Veneer boards: Veneer—MDF support board—veneer
MDF under decorative paper
MDF under surface coating
HDF for laminate floors, parquet floors
Insulation boards, for example, heat and footfall sound insulation boards
LDF boards
Molded parts, for example, in the sanitary area
Moldings, for example, for automobile internal paneling
